(19) United States
(12) Patent Application Publication
(10) Pub. No.: US 2015/0182525 A1
(43) Pub. Date: Jul. 2, 2015

(54) 4-Amino-5-Fluoro-3-[6-(4-Methylpiperazin-1-YL)-1H-Benzimidazol-2-YL]-1H-Quinolin-2-one for use in the Treatment of Adenoid Cystic Carcinoma

(71) Applicants: Michael Shi, Chalfont, PA (US); Michael Wick, Helotes, TX (US)

(72) Inventors: Michael Shi, Chalfont, PA (US); Michael Wick, Helotes, TX (US)

(73) Assignee: Novartis AG, Basel (CH)

(21) Appl. No.: 14/117,057

(22) PCT Filed: May 18, 2012
(86) PCT No.: PCT/US12/38490

§ 371 Date: Nov. 12, 2013

Related U.S. Application Data
(60) Provisional application No. 61/487,939, filed on May 19, 2011.

(51) Int. Cl. A61K 31/496; A61K 31/337

(52) U.S. Cl. CPC A61K 31/496; A61K 31/337

(57) ABSTRACT

The present invention describes a method of reducing solid tumors in a subject having an adenoid cystic carcinoma comprising administering a therapeutically effective amount of 4-amino-5-fluoro-3-[6-(4-methylpiperazin-1-yl)-1H-benzimidazol-yl]-1H-quinolin-2-one or a pharmaceutically acceptable salt thereof.

### FIELD OF THE INVENTION

The present invention relates to the use of a certain compound that inhibits fibroblast growth factors (FGFs) and their receptors (FGFRs) for the treatment of adenoid cystic carcinoma. In particular, the present invention is directed to a method for treating adenoid cystic carcinomas, e.g. via the FGF/FGFR pathway, using 4-amino-5-fluoro-3-[6-(4-methylpiperazin-1-yl)-1H-benzimidazol-2-yl]-1H-quinolin-2-one or a tautomer thereof, e.g. 4-amino-5-fluoro-3-[5-(4-methylpiperazin-1-yl)-1H-benzimidazol-2-yl]-1H-quinolin-2-one or a pharmaceutically acceptable salt thereof.

### BACKGROUND OF THE INVENTION

Fibroblast growth factors (FGFs) and their receptors (FGFRs) are a highly conserved group of proteins with instrumental roles in angiogenesis, vasculogenesis, and wound healing, as well as tissue patterning and limb formation in embryonic development. FGFs and FGFRs affect cell migration, proliferation, and survival, providing wide-ranging impacts on health and disease.

The FGFR family comprises four major types of receptors, FGFR1, FGFR2, FGFR3, and FGFR4. These receptors are transmembrane proteins having an extracellular domain, a transmembrane domain, and an intracytoplasmic domain. Each of the extracellular domains contains either two or three immunoglobulin (Ig) domains. Some FGFRs exist in different isoforms which differ in specific segments of the molecule, such as FGFR-IIIb and FGFR1-IIIc, which differ in the C-terminal region of the third Ig domain. Transmembrane FGFRs are monomeric tyrosine kinase receptors, activated by dimerization, which occurs at the cell surface in a complex of FGFR dimers, FGF ligands, and heparin glycans or proteoglycans. Extracellular FGFR activation by FGF ligand binding to an FGFR initiates a cascade of signaling events inside the cell, beginning with the receptor tyrosine kinase activity.

U.S. Pat. No. 7,678,890 discloses FGFR fusion proteins and that FGFR1, and FGFR3, and/or FGFR4 are often over-expressed in cancer. FGFR1 is over-expressed in leukemia, including B-cell acute lymphoblastic leukemia, chronic myelomonocytic leukemia, chronic lymphocytic leukemia, and chronic myeloid leukemia; in lymphoma, including Hodgkin's lymphoma, non-Hodgkin's lymphoma, and extranodal lymphoma; in myeloma, including plasmacytoma; in sarcoma, including malignant neoplasms of the bone and soft tissues; in neurologic cancer, including malignant neoplasms of the brain; in breast cancer, including malignant neoplasms of the female breast; in digestive tract/gastrointestinal cancer, including malignant neoplasms of the ampulla of Vater, appendix, colon, duodenum, esophagus, liver, pancreas, peritoneum, rectum, small intestine, and stomach; in endocrine cancer, including malignant neoplasms of the adrenal gland, islets of Langerhans, and thyroid gland; in eye cancer, including malignant neoplasms of the eye; in genitourinary cancer, including malignant neoplasms of the bladder, kidney, prostate, and testis; in gynecologic cancer, including malignant neoplasms of the uterine cervix, myometrium, ovary, uterus, endometrium, placenta, and vulva; in head and neck cancer, including malignant neoplasms of the larynx, salivary gland, nasal cavity, oral cavity, parotid gland, and tongue; in respiratory/thoracic cancer, including malignant neoplasms of the lung, thymus, and trachea; and in skin cancer.

FGFR3 is over-expressed in lymphoma, including Burkitt's lymphoma; in sarcoma, including malignant neoplasms of the bone and soft tissues; in neurologic cancer, including malignant neoplasms of the brain; in breast cancer, including malignant neoplasms of the female breast and male breast; in digestive tract/gastrointestinal cancer, including malignant neoplasms of the ampulla of Vater, colon, duodenum, esophagus, gallbladder, liver, pancreas, rectum, small intestine, and stomach; in endocrine cancer, including malignant neoplasms of the islets of Langerhans and thyroid gland; in genitourinary cancer, including malignant neoplasms of the bladder, kidney, prostate, testis, and ureter; in gynecologic cancer, including malignant neoplasms of the uterine cervix, ovary, uterus, endometrium, and vulva; in head and neck cancer, including malignant neoplasms of the larynx, oral cavity, parotid gland, tongue, and tonsil; in respiratory/thoracic cancer, including malignant neoplasms of the lung; and in skin cancer.

FGFR4 is over-expressed in lymphoma, including non-Hodgkin's lymphoma; in sarcoma, including malignant neoplasms of the bone, heart, and soft tissues; in breast cancer, including malignant neoplasms of the female breast; in digestive tract/gastrointestinal cancer, including malignant neoplasms of the colon, duodenum, esophagus, gallbladder, liver, pancreas, rectum, small intestine, and stomach; in endocrine cancer, including malignant neoplasms of the adrenal gland and islets of Langerhans; in genitourinary cancer, including malignant neoplasms of the kidney and testis; in gynecologic cancer, including malignant neoplasms of the ovary and endometrium; in head and neck cancer, including malignant neoplasms of the parotid gland; in respiratory/thoracic cancer, including malignant neoplasms of the lung; and in skin cancer.

Adenoid cystic carcinomas (ACC) are aggressive, although slow growing cancers with poor prognosis. ACC proliferates in salivary glands found in the neck and head and exocrine glands found in the breast, cervix, vulva and tracheobronchial tree. Despite an identified recurrent and tumor specific t(6;9) translocation in ACC of the head and neck, which is associated with the transcription factor genes MYB and NFIB, the molecular pathogenesis was poorly understood prior to the present invention. Evidence in support of any systemic therapy for metastatic adenoid cystic carcinoma is limited and no single pharmaceutical agents or combinations of pharmaceutical agents having predictable and significant impact on this tumor have been disclosed. Thus there is still an unmet need for patients having adenoid cystic carcinomas.

The compound 4-amino-5-fluoro-3-[6-(4-methylpiperazin-1-yl)-1H-benzimidazol-2-yl]-1H-quinolin-2-one, also referred to as dovitinib, or a pharmaceutically acceptable salt thereof, of formula I

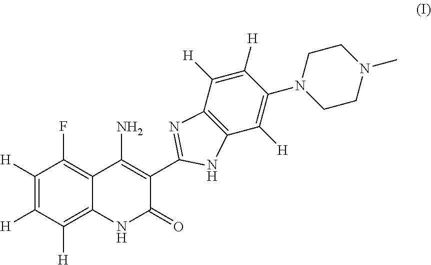 [SMILES: CN1CCN(c2ccc3nc(-c4c(N)c5c(F)cccc5[nH]c4=O)[nH]c3c2)CC1]

inhibits certain protein kinases, such as tyrosine receptor kinases (RTKs). Compound I, a tautomer thereof or a pharmaceutically acceptable salts, including the mono-lactic acid salt, are described in U.S. Patent Nos. 6,605,617, 6,774,237, 7,335,774, and 7,470,709, and in U.S. patent application Ser. Nos. 10/982,757, 10/982,543, and 10/706,328, and in the published PCT applications WO 2006/127926 and WO2009/115562. Using adenoid cystic carcinoma specific (ACC) xenograft models validated by histology as having morphology of ACC primary tumors, it was shown that dovitinib was effective for inhibiting tumor growth in primary ACC xenografts.

### SUMMARY OF THE INVENTION

The present invention provides a method for treating adenoid cystic carcinoma resulting from deregulation of fibroblast growth factor receptor FGFR1, in a subject in need thereof comprising administering a therapeutically effective amount of 4-amino-5-fluoro-3-[6-(4-methylpiperazin-1-yl)-1H-benzimidazol-2-yl]-1H-quinolin-2-one, or a tautomer thereof, or a pharmaceutically acceptable salt thereof.

The present invention provides a method for treating adenoid cystic carcinoma in a subject in need thereof comprising administering a therapeutically effective amount of 4-amino-5-fluoro-3-[6-(4-methylpiperazin-1-yl)-1H-benzimidazol-2-yl]-1H-quinolin-2-one or a pharmaceutically acceptable salt thereof.

The present invention provides a method of reducing solid tumor in a subject having an adenoid cystic carcinoma comprising administering a therapeutically effective amount of 4-amino-5-fluoro-3-[6-(4-methylpiperazin-1-yl)-1H-benzimidazol-2-yl]-1H-quinolin-2-one or a pharmaceutically acceptable salt thereof.

The present invention also provides the use of 4-amino-5-fluoro-3-[6-(4-methylpiperazin-1-yl)-1H-benzimidazol-2-yl]-1H-quinolin-2-one or a pharmaceutically acceptable salt thereof and a pharmaceutically acceptable carrier for the preparation of a medicament for the treatment of adenoid cystic carcinoma mediated by fibroblast growth factor receptor FGFR1.

The present invention provides 4-amino-5-fluoro-3-[6-(4-methylpiperazin-1-yl)-1H-benzimidazol-2-yl]-1H-quinolin-2-one or a tautomer thereof or a pharmaceutically acceptable salt thereof for use in the treatment of an adenoid cystic carcinoma in a subject in need thereof, wherein the adenoid cystic carcinoma is located in salivary and lacrimal glands of the head and neck, in glands of the larynx, in the bronchial tree in the lung, in mammary glands in the breast, in ovarian ducts and Bartholin's glands in the vulva.

The present invention pertains to 4-amino-5-fluoro-3-[6-(4-methylpiperazin-1-yl)-1H-benzimidazol-2-yl]-1H-quinolin-2-one, or a tautomer thereof or a pharmaceutically acceptable salt thereof, for use in the treatment of adenoid cystic carcinomas.

The present invention pertains to the use of 4-amino-5-fluoro-3-[6-(4-methylpiperazin-1-yl)-1H-benzimidazol-2-yl]-1H-quinolin-2-one or a pharmaceutically acceptable salt thereof for the preparation of a medicament for the treatment of adenoid cystic carcinoma.

The present invention pertains to 4-amino-5-fluoro-3-[6-(4-methylpiperazin-1-yl)-1H-benzimidazol-2-yl]-1H-quinolin-2-one or a pharmaceutically acceptable salt thereof for use in the treatment or prevention of progression of ACC wherein said compound is the sole active ingredient used for the treatment or prevention of progression of said indication.

According to the present invention, 4-amino-5-fluoro-3-[6-(4-methylpiperazin-1-yl)-1H-benzimidazol-2-yl]-1H-quinolin-2-one can be in the lactacte salt form thereof, for example in the mono lactate form.

The present invention pertains to a combination of 4-amino-5-fluoro-3[6-(4-methylpiperazin-1-y1)-1H-benzimidazol-2-yl]-1H-quinolin-2-one, or a tautomer thereof, or a pharmaceutically acceptable salt thereof, and docetaxel. According to the present invention, said combination can be used in the treatment of ACC, for example ACC of the salivary and lacrimal glands of the head and neck, in glands of the larynx, in the bronchial tree in the lung, in mammary glands in the breast, in ovarian ducts and Bartholin's glands in the vulva.

According to the present invention, 4-amino-5-fluoro-3-[6-(4-methylpiperazin-1-yl)-1H-benzimidazol-2-yl]-1H-quinolin-2-one, for example the lactate salt form, is administered as follows: 500 mg per day for 5 days on followed by two days off treatment on a weekly basis.

### BRIEF DESCRIPTION OF THE DRAWINGS

FIG. 1 summarizes a gene expression correlation of ACC primary tumors and corresponding ACC xenografts.

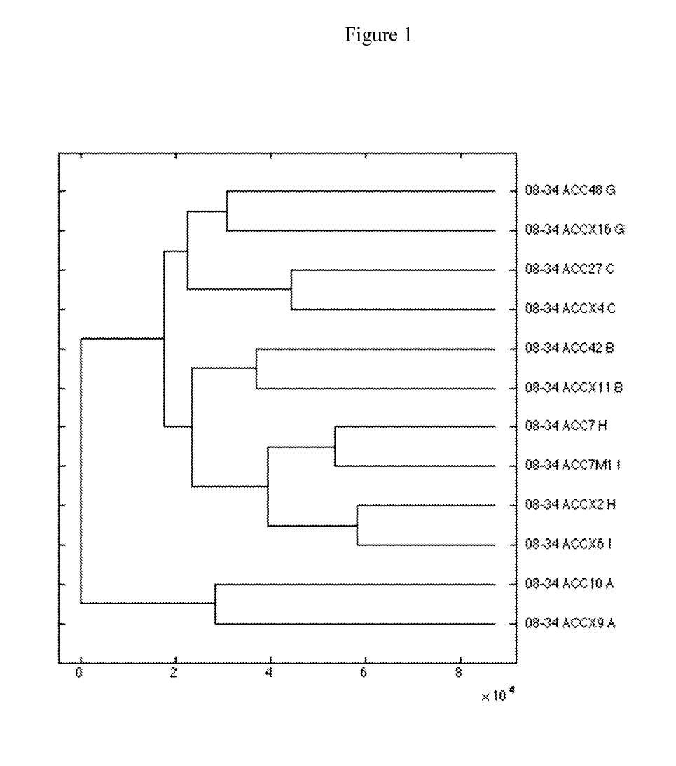

FIG. 2 summarizes FGFR1 phosphopeptides detected, all increased in ACC compared to normal salivary gland by Phosphoscan^{TM} analysis.

FIG. 3 summarizes microarray gene expression data from FGFR1 genes as a function of FGFR1 transcript in ACC primary tumors as compared to benign salivary gland tissue.

FIG. 4 summarizes Western blot analysis that indicates FGFR1 phosphorylation occurs in ACC tumors but not in normal salivary glands.

FIG. 5 summarizes Western blot analysis of corresponding low passage ACC xenografts confirmed FGFR1 expression and constitutive phosphorylation at Tyr 6534.

FIG. 6 summarizes tumor growth of an ACC xenograft in nude mice up to Day 38 when treated with dovitinib, docetaxel and the combination of both.

FIG. 7 summarizes tumor growth of an ACC xenograft in nude mice up to Day 38 when treated with dovitinib, docetaxel and the combination of both.

FIG. 8 summarizes radiologic imaging data of an ACC target lesion (facial) before and after dovitinib treatment in an ACC patient, indicating a 70% reduction in tumor diameter after 1 cycle of dovitinib treatment.

### DETAILED DESCRIPTION OF THE INVENTION

The present invention pertains to a method for treating adenoid cystic carcinoma resulting from deregulation of fibroblast growth factor receptor FGFR1 in a subject in need thereof comprising administering a therapeutically effective amount of 4-amino-5-fluoro-3-[6-(4-methylpiperazin-1-yl)-1H-benzimidazol-2-yl]-1H-quinolin-2-one or a pharmaceutically acceptable salt thereof.

In one embodiment, a method is provided for treating adenoid cystic carcinoma in a subject in need thereof comprising administering a therapeutically effective amount of 4-amino-5-fluoro-3-[6-(4-methylpiperazin-1-yl)-1H-benzimidazol-2-yl]-1H-quinolin-2-one or a pharmaceutically acceptable salt thereof.

In a separate embodiment a method is provided for treating adenoid cystic carcinoma in a subject in need thereof comprising administering a therapeutically effective amount of a compound selected from BGJ398 (Novartis), ponatinib (AP-24534), ARQ-087, E-3810, KI23057 and FP-1039 (FGF trap), or a pharmaceutically acceptable salt thereof.

In a separate embodiment a method is provided for treating adenoid cystic carcinoma in a subject in need thereof comprising administering a therapeutically effective amount of 4-amino-5-fluoro-3-[6-(4-methylpiperazin-1-yl)-1H-benzimidazol-2-yl]-1H-quinolin-2-one or a pharmaceutically acceptable salt thereof in combination with a compound selected from BGJ398 (Novartis), ponatinib (AP-24534), ARQ-087, E-3810, KI23057 and FP-1039 (FGF trap), or a pharmaceutically acceptable salt thereof.

Adenoid cystic carcinoma according to the present invention refers to adenoid cystic carcinoma of the glands, for example from the glands selected from salivary and lacrimal glands of the head and neck, glands of the larynx, the bronchial tree in the lung, mammary glands in the breast, ovarian ducts, and Bartholin's glands in the vulva.

The present invention pertains to a combination of 4-amino-5-fluoro-3-[6-(4-methylpiperazin-1 -y1)-1H-benzimidazol-2-yl]-1H-quinolin-2-one, a tautomer thereof or a pharmaceutically acceptable salt thereof and docetaxel for use in the treatment of adenoid cystic carcinoma.

The present invention also pertains to 4-amino-5-fluoro-3-[6-(4-methylpiperazin-1-yl)-1H-benzimidazol-2-yl]-1H-quinolin-2-one, a tautomer thereof or a pharmaceutically acceptable salt thereof a sole active ingredient for the treatment of adenoid cystic carcinoma.

Ninety percent of patients presenting diagnosed ACC have an identified recurrent and tumor specific t(6;9) translocation in ACC of the head and neck, which is associated with the transcription factor genes MYB and NFIB, however, the molecular pathogenesis was poorly understood prior to the present invention. ACC xenografts having a characteristic fusion gene and histologically validated were used in accordance with the invention. The ACC xenografts exhibit histological features of primary ACC tumors, retaining typical ACC morphology through multiple passages (data not shown). ACC xenografts are similar to corresponding ACC primary tumors in gene expression (Am. J. Path. 161, 1315-1323 (2002)) and as summarized for exemplary ACC xenografts used in accordance with the invention (data not shown).

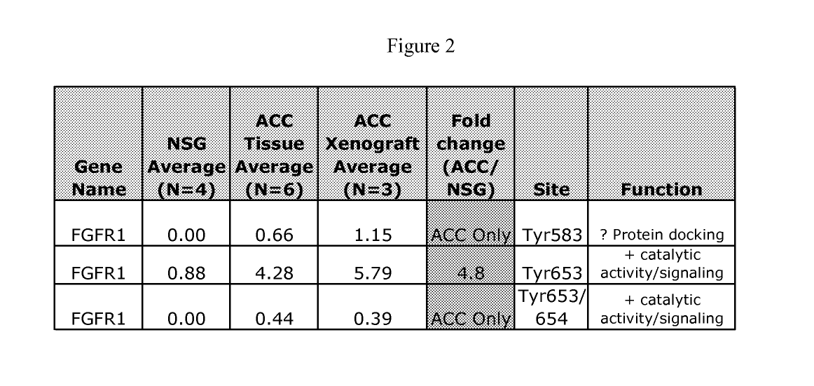

A total of 6 primary ACC tumors, 3 ACC xenografts and 4 normal salivary glands (NSG) were compared using cell signaling phosphopeptide analysis (Phosphoscan™). Analysis of phosphotyrosine residues indicated 1092 discrete phosphopeptides were detected. It was shown that a subset of phosphopeptides associated with FGFR1 genes were phosphorylated 2 times greater in ACC tumors as compared to NSG. A total of 3 FGFR1 phosphopeptides detected, all increased in ACC compared to normal salivary gland (FIG. 2).

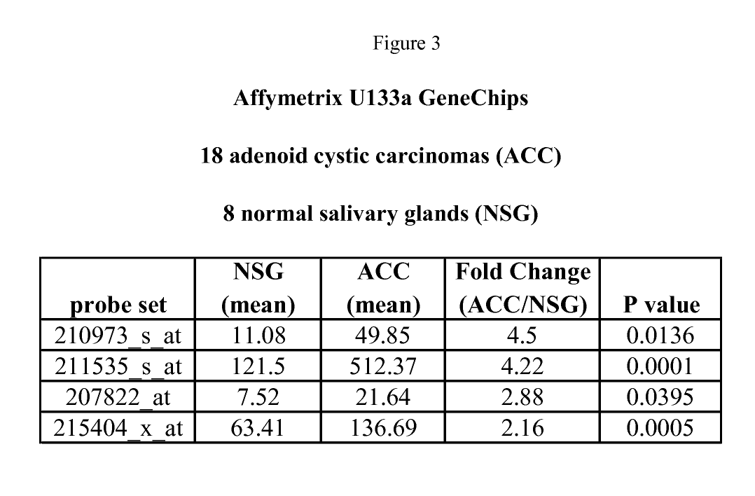

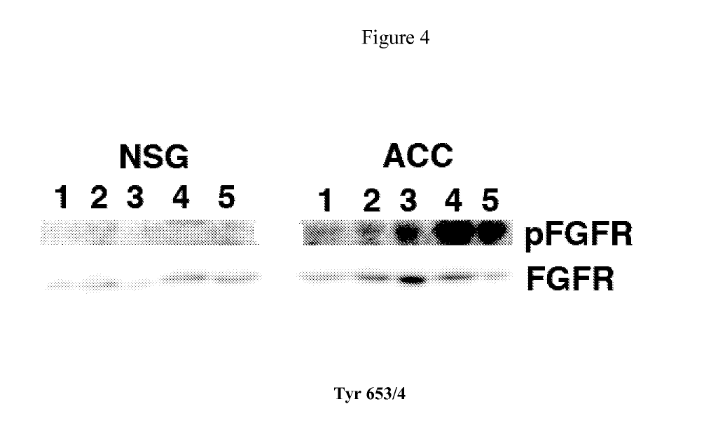

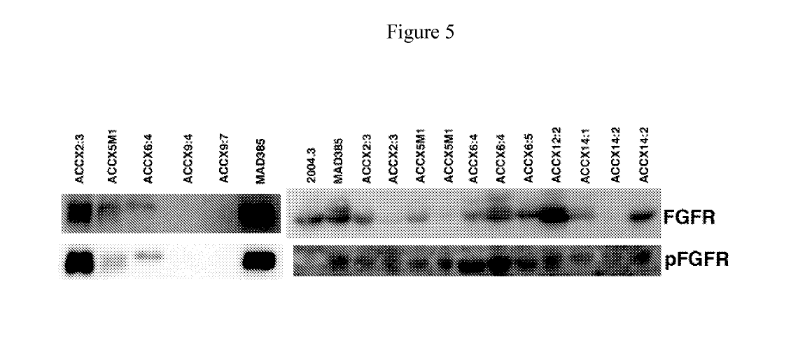

Microarray gene expression data from four probe sets for FGFR1 genes exhibited statistically significant increases in FGFR1 transcript in ACC primary tumors as compared to benign salivary gland tissue (FIG. 3). Western blot analysis indicated FGFR1 phosphorylation occurs in ACC tumors but not in normal salivary glands (FIG. 4). Western blot analysis of corresponding low passage ACC xenografts confirmed FGFR1 expression and constitutive phosphorylation at Tyr 6534 (FIG. 5). It was determined that FGFR1 was not mutated in ACC tumors.

Based on studies describing the relationship between MYB and FGFR (MYB upregulates FGF2/bFGF expression in melanoma cells, Cell Growth Diff 8:1199, 1997; MYB upregulates FGF4 expression in HeLa cells, J Biol Chem 277:4088, 2002; FGFR1 signaling cooperates with MYB in primitive erythroid precursors to maintain proliferation and suppress differentiation, Oncogene 21:400, 2002 and the knowledge there is a MYB-response element in the FGFR1 promoter region, it was determined that ACC xenografts represented an excellent model of activation and cooperation.

ACC xenografts: Tissue from donor models was implanted in immunodeficient mice and tumor growth was followed until an endpoint was reached at which point a sample was sent for histologic confirmation of tissue type and origin. Once confirmed, established ACC xenograft models were developed until growth characteristic stabilized at which point viable stocks were collected and banked. According to one embodiment, the efficacy of dovitinib was evaluated in inhibiting tumor growth in certain ACC xenografts.

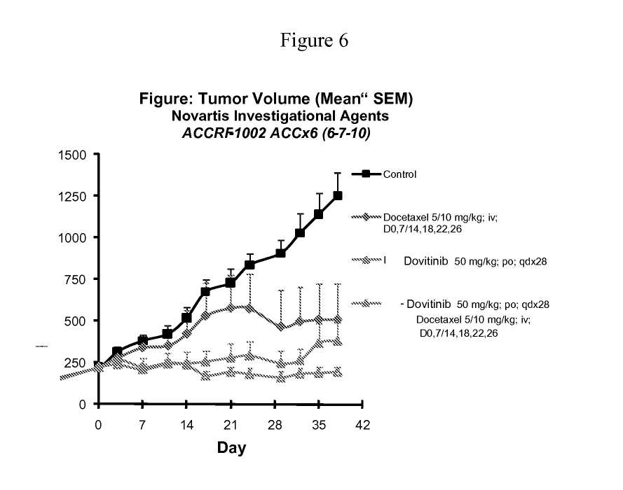

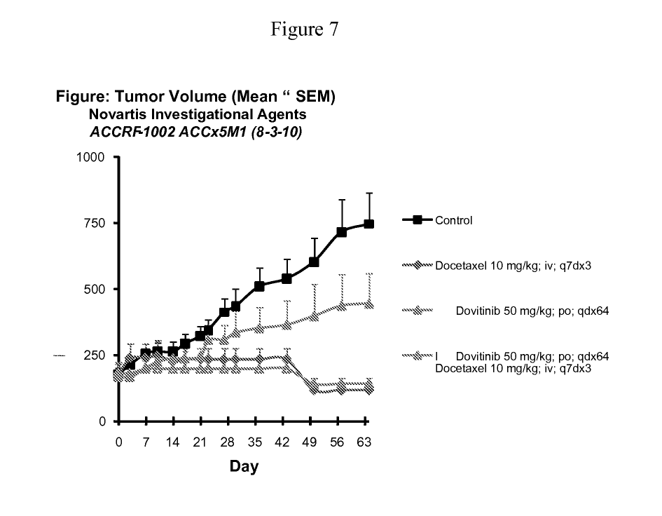

It was shown that dovitinib was surprisingly effective in inhibiting tumor in a number of primary ACC xenografts (FIGS. 6 and 7).

It was further shown that dovitinib was surprisingly effective in reducing ACC tumor growth in a human clinical trial, even after 1 cycle of treatment.

Specific embodiments of the invention will now be demonstrated by reference to the following examples. It should be understood that these examples are disclosed solely by way of illustrating the invention and should not be taken in any way to limit the scope of the present invention.

### Example 1

The ACCx6 xenograft tumor line is derived from adenoid cystic carcinoma donor models implanted in immunodeficient mice. The tumors are maintained by engraftment in nude mice. A 1 mm³ fragment is implanted subcutaneously in the right flank of each test animal The tumors are measured with calipers twice weekly, and daily as the mean volume approached 100-150 mm³ Seven days after tumor cell implantation, on D1 (day 1) of the study, the animals are sorted into groups of ten mice, with individual tumor sizes of 100-1250 mm³ Tumor size, in mm³, is calculated from

$$Tumor volume = \frac{{w}^{2} \times 1}{2}$$

where “w” is the width and “1” is the length, in mm, of the tumor. Tumor weight is estimated with the assumption that 1 mg is equivalent to 1 mm³ of tumor volume. For the efficacy study dovitinib and its vehicle are each administered orally (p.o.), once daily for twenty eight consecutive days (qd x28). Docetaxel is administered i.v., once daily on alternate days for five doses (qod x5). All drugs in combination are administered within 30-60 minutes. The dosing volume, 10 mL/kg (0.2 mL20 g mouse), is scaled to the weight of each animal as determined on the day of dosing, except on weekends when the previous BW was carried forward.

The study begins on Day 1 (D1). Efficacy is determined from tumor volume changes up to D28 (day 28). Efficacy is determined on D28.

For the purpose of statistical and graphical analyses, ΔTV, the difference in tumor volume between D1 (the start of dosing) and the endpoint day, was determined for each animal For each treatment group, the response on the endpoint day was calculated by the following relation:

TC(%)=100×ΔT/ΔC, for ΔT>0 where

ΔT=(mean tumor volume of the drug-treated group on the endpoint day)−(mean tumor volume of the drug-treated group on D1), and AC=(mean tumor volume of the control group on the endpoint day)−(mean tumor volume of the control group on D1).

A treatment that achieved a TC value of 40% or less was classified as potentially therapeutically active.

FIG. 7 shows the treatment response up to Day 28. (n) is the number of animals in a group not dead from treatment-related, accidental, or unknown causes. The Mean Volume is the group mean tumor volume; The Change is the difference between D1 and D28. TC is 100×(ΔT/ΔC) which is the percent change between Day 1 and Day 28 in the mean tumor volume of treated group (ΔT) compared with change in control group (ΔV).

Statistical significance is shown by Kruskal-Wallis with post hoc Dunn's multiple comparison test): ns=not significant; *=p<0.05; **=p<0.01; and ***=p<0.0001, compared to the indicated group. Monotherapy with 50 mg/kg dovtinib resulted in significant median growth inhibition (P<0.001) as compared to 5/10 mg/kg docetaxel monotherapy. Combination therapy of dovitinib and docetaxel provided significant (P<0.01) improvements over dovitinib and docetaxel monotherapies, respectively.

### Example 2

The ACCx5M1 xenograft tumor line is derived from adenoid cystic carcinoma donor models implanted in immunodeficient mice. The tumors are maintained by engraftment in nude mice. A 1 mm³ fragment is implanted subcutaneously in the right flank of each test animal The tumors are measured with calipers twice weekly, and daily as the mean volume approached 100-150 mm³ Seven days after tumor cell implantation, on D1 (day 1) of the study, the animals are sorted into groups of ten mice, with individual tumor sizes of 100-750 mm³ Tumor size, in mm³, is calculated from

$$Tumor volume = \frac{{w}^{2} \times 1}{2}$$

where “w” is the width and “1” is the length, in mm, of the tumor. Tumor weight is estimated with the assumption that 1 mg is equivalent to 1 mm³ of tumor volume.

For the efficacy study dovitinib and its vehicle are each administered orally (p.o.), once daily for sixty four consecutive days (qd x64). Docetaxel is administered i.v., once daily on alternate days for five doses (qod x5). All drugs in combination are administered within 30-60 minutes. The dosing volume, 10 mL/kg (0.2 mL/20 g mouse), is scaled to the weight of each animal as determined on the day of dosing, except on weekends when the previous BW was carried forward.

The study begins on Day 1 (D1). Efficacy is determined from tumor volume changes up to D64 (day 64). For the purpose of statistical and graphical analyses, ΔTV, the difference in tumor volume between D1 (the start of dosing) and the endpoint day, was determined for each animal For each treatment group, the response on the endpoint day was calculated by the following relation:

TC(%)=100×ΔT/ΔC, for ΔT>0 where ΔT=(mean tumor volume of the drug-treated group on the endpoint day)−(mean tumor volume of the drug-treated group on D1), and ΔC=(mean tumor volume of the control group on the endpoint day)−(mean tumor volume of the control group on D1).

A treatment that achieved a T/C value of 50% or less was classified as potentially therapeutically active. Each animal was euthanized when its neoplasm reached the endpoint volume (800 mm3), or on the last day of the study (D64). For each animal whose tumor reached the endpoint volume, the time to endpoint (TTE) was calculated by the following equation:

$$TTE = \frac{log 10 ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size.

An animal with a tumor that did not reach the endpoint is assigned a TTE value equal to the last day. An animal classified as having died from treatment-related (TR) causes or non-treatment-related metastasis (NTRm) is assigned a TTE value equal to the day of death. An animal classified as having died from non-treatment-related (NTR) causes is excluded from TTE calculations.

Treatment efficacy was determined from tumor growth delay (TGD), which is defined as the increase in the median TTE for a treatment group compared to the control group: TGD=T C, expressed in days, or as a percentage of the median TTE of the control group:

$$\% TGD = \frac{T - C}{C} \times 100$$

where T is the median TTE for a treatment group and C is TTE for control group 1.

Treatment efficacy may also be determined from the tumor volumes of animals remaining in the study on the last day, and from the number of regression responses. The MTV(n) is defined as the median tumor volume on D64 in the number of animals remaining, n, whose tumors had not attained the endpoint volume.

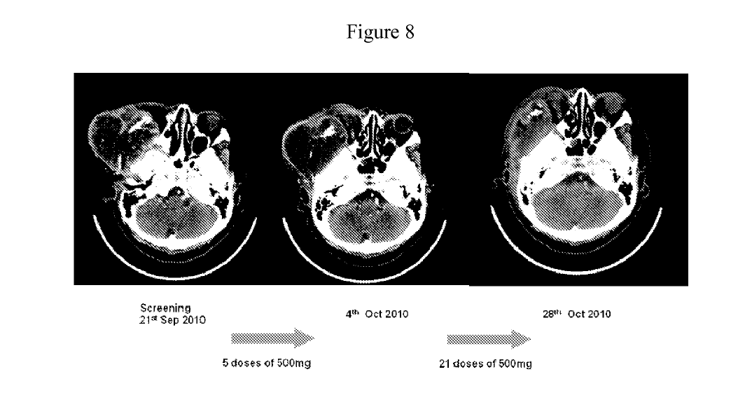

Treatment may cause partial regression (PR) or a complete regression (CR) of the tumor in a animal A PR indicates that the tumor volume is 50% or less of its D1 volume for three consecutive measurements during the course of the study, and equal to or greater than 13.5 mm3 for one or more of these three measurements. A CR indicates that the tumor volume was less than 13.5 mm³ for three consecutive measurements during the course of the study. An animal with a CR at the termination of a study is additionally classified as a tumor-free survivor (TFS). p FIG. 8 shows the treatment response up to the study endpoint (D64, day 64 or tumor volume of 750 mm³ which ever comes first). The statistical significance is analysed by the Logrank test: ns=not significant; *=p<0.05; **=p<0.01; and ***=p<0.0001, compared to the indicated group. MTV (n) is the median tumor volume (mm³) for the number of animals on the day of TGD analysis (excludes animals with tumor volume at endpoint).

Monotherapy with 50 mg/kg dovitinib resulted in a % TGD of 49. The survival extension was significant (P<0.05). The combination of dovitinib and docetaxel significantly improved upon the corresponding dovitinib monotherapy (P<0.05) and the corresponding docetaxel monotherapy (P<0.001).

### Example 3

### Treatment of Adenoid Cystic Carcinoma Patient with Dovitinib

A 52 year old female patient with Stage IV adenoid cystic carcinoma was enrolled in a dovitinib clinical trial.

The patient was originally diagnosed 20 years ago with Stage II well differentiated ACC on the right cheek mucosa. Patient was previously treated using tumor resection of right cheek mucosa and right upper jaw at age 30 and treated with radiation therapy, followed by chemotherapy with 5 FU (250 mg/m²), ciplatin (40 mg/m²), doxorubicin (20 or 27 mg/m²) and cyclophosphamide (400 mg/m²).

Recurrence of ACC occurred at age 44, and patient was again treated using surgery of right cheek and submandibular Lymph nodes. Patient was subsequently treated with radiation therapy and followed by chemotherapy (TS-1, 120 mg/day).

Patient was enrolled into the dovitinib clinical trial and was treated with dovitinb 500 mg 5 days on 2 days off. After 1 cycle of treatment (4 weeks), there was a 70% reduction of tumor diameter on the right facial target lesion and 26% reduction of total diameter of target lesions, as summarized in FIG. 8. The radiological imaging data from the human patient clinical trials clearly and unambiguously indicate the effectiveness of dovitinib in reducing tumor growth in patients presenting ACC.

1. 4-amino-5-fluoro-3-[6-(4-methylpiperazin-1-yl)-1H-benzimidazol-2-yl]-1H-quinolin-2-one or a tautomer or a pharmaceutically acceptable salt therof for use in the treatment of adenoid cystic carcinoma.

2. A method of reducing solid tumors or preventing the progression solid tumors in a subject having an adenoid cystic carcinoma comprising administering a therapeutically effective amount of 4-amino-5-fluoro-3-[6-(4-methylpiperazin-1-yl)-1H-benzimidazol-2-yl]-1H-quinolin-2-one or a pharmaceutically acceptable salt thereof in combination with docetaxel or a pharmaceutically acceptable salt thereof.

3. Use of 4-amino-5-fluoro-3-[6-(4-methylpiperazin-1-yl)-1H-benzimidazol-2-yl]-1H-quinolin-2-one or a pharmaceutically acceptable salt thereof and a pharmaceutically acceptable carrier of the preparation of a medicament for treating solid tumors in a subject having an adenoid cystic carcinoma.

4. Combination of 4-amino-5-fluoro-3-[6-(4-methylpiperazin-1-yl)-1H-benzimidazol-2-yl]-1H-quinolin-2-one or a tautomer or a pharmaceutically acceptable salt thereof and docetaxel for use in the treatment of adenoid cystic carcinoma.

5. Method of reducing solid tumors in a subject having adenoid cystic carcinoma comprising administering a therapeutically effective amount of 4-amino-5-fluoro-3-[6-(4-methylpiperazin-1-yl)-1H-benzimidazol-2-yl]-1H-quinolin-2-one or a tautomer or a pharmaceutically acceptable salt thereof in combination with docetazel or a pharmaceutically acceptable salt thereof.

6. Use of a combination according to claim 4 for the preparation of a medicament for treating or preventing growth of solid tumors in a patient having an adenoid cystic carcinoma.

7. 4-amino-5-fluoro-3-[6-(4-methylpiperazin-1-yl)-1H-benzimidazol-2-yl]-1H-quinolin-2-one or a tautomer or a pharmaceutically acceptable salt thereof according to claim 1, combination according to claim 4, method according to claim 2 or 5, or use according to claim 3 or 6 wherein the adenoid cystic carcinoma is an adenoid cystic carcinoma of glands selected from: salivary and lacrimal glands of the head and neck, glands of the larynx, the bronchial tree in the lung, mammary glands in the breast, ovarian ducts, Bartholin's glands in the vulva and combinations thereof.

8. 4-amino-5-fluoro-3-[6-(4-methylpiperazin-1-yl)-1H-benzimidazol-2-yl]-1H-quinolin-2-one or a tautomer or a pharmaceutically acceptable salt thereof according to claim 1 wherein 4-amino-5-fluoro-3-[6-(4-methylpiperazin-1-yl)-1H-benzimidazol-2-yl]-1H-quinolin-2-one or a tautomer or a pharmaceutically acceptable salt thereof is administered to the patient on weekly basis as 500 mg per day for 5 days followed by 2 days off treatment.
